(19) United States
(12) Patent Application Publication
(10) Pub. No.: US 2021/0009678 A1
(43) Pub. Date: Jan. 14, 2021

(54) METHODS AND COMPOSITIONS FOR ALLERGIC DISORDERS

(71) Applicant: North Carolina State University, Raleigh, NC (US)

(72) Inventors: Bruce Hammerberg, Raleigh, NC (US); Thierry Olivry, Riga (LV)

(21) Appl. No.: 16/982,759

(22) PCT Filed: Mar. 22, 2019
(86) PCT No.: PCT/US2019/023529

§ 371 Date: Sep. 21, 2020

Related U.S. Application Data
(60) Provisional application No. 62/647,511, filed on Mar. 23, 2018.

(51) Int. Cl. C07K 16/24; C07K 16/28; A61P 37/08

(52) U.S. Cl. CPC C07K 16/244; C07K 16/2866; A61K 2039/505; C07K 2317/76; A61P 37/08

(57) ABSTRACT

The present invention relates to methods and compositions for treating, reducing, delaying, and/or preventing flares (e.g., recurrences, relapses) of allergic disorders with a biologic agent.

### STATEMENT OF PRIORITY

This application claims the benefit, under 35 U.S.C. § 119(e), of U.S. Provisional Application Ser. No. 62/647,511, filed Mar. 23, 2018, the entire contents of which are incorporated by reference herein.

### FIELD OF THE INVENTION

The present invention is directed to methods of treating allergic disorders and/or preventing their recurrence with a biologic agent. In particular, the biologic agent is directed toward targeting inflammatory mediators of the early phase of the inflammatory cascade.

### BACKGROUND OF THE INVENTION

Allergic disorders are currently the 6th leading cause of chronic illness in the U.S. and are steadily increasing every year. Studies have shown that as many as 30% of adults and 40% of children in the U.S. are currently afflicted with an allergic disorder. Allergic disorders also account for about 25% of all visits to the veterinarian for dogs and cats. For example, canine atopic dermatitis in dogs is the second most common allergic skin condition affecting about 10-15% of animals.

Allergic disorders in humans as well as dogs and cats have been primarily linked to the exposure to specific allergens, such as dust mites, pollen, dander, latex, insect venoms, medicines or certain foods (e.g., milk, meats, chicken, peanuts, eggs, and shellfish). Medications currently prescribed for treating allergic disorders vary depending on the allergic disorder and corresponding symptoms being treated. For example, antihistamines target the H₁ histamine receptor and are a mainstay of treatment for allergic rhinitis but have been of limited value in asthma. Asthma is generally treated with inhaled corticosteroids (which suppress many of the pathways that contribute to inflammation) and agonists of (3-adrenergic receptors (which induce bronchodilation) but prolonged use of corticosteroids can result in the development of undesirable side effects. In addition, not all treatment options are equally effective in every patient.

Thus, due to the variability in efficacy of the same pharmacological agent in treating various allergic disorders and/or the variable clinical responses of patients to the same pharmacological agent, there remains great need in developing better therapeutic agents for treating allergic disorders. In particular, treatment options that are able to prevent relapses, delays, and/or recurrences of inflammatory events would be highly desirable.

The present invention overcomes previous shortcomings in the art by providing methods and compositions for treatment of allergic disorders by targeting inflammatory mediators of the early phase of an inflammatory cascade, optionally in a subject for whom signs of the allergic disorder are controlled.

### SUMMARY OF THE INVENTION

The current invention relates to a method of preventing/reducing/inhibiting an allergic disorder in a subject in need thereof, the method comprising administering to the subject a composition comprising a therapeutically effective amount of a biologic agent targeting an inflammatory mediator during early phase inflammation, with the proviso that the biologic agent is not an anti-IgE antibody. In some embodiments, the subject is asymptomatic or paucisymptomatic with or without currently receiving and/or having received an anti-inflammatory agent, an anti-allergy agent, an immunomodulatory agent or a combination thereof. In some embodiments, a paucisymptomatic subject may be clear or almost clear of symptoms and/or signs of the allergic disorder. In some embodiments, the anti-inflammatory agent, the anti-allergy agent, and/or the immunomodulatory agent is selected from a glucocorticoid, a non-steroidal anti-inflammatory agent, a leukotriene antagonist, a JAK inhibitor, an immunoglobulin, an anti-histamine, and combinations thereof administered by either oral, subcutaneous, intramuscular, intravenous and/or topical routes. In some embodiments, the subject is a canine, feline, equine, or human. In some embodiments, the subject is a human, dog, cat, or horse. In some embodiments, the inflammatory mediator is selected from histamine, a leukotriene, a prostaglandin, a cytokine, platelet activating factor (PAF), a growth factor, a protease, an immunoglobulin, and combinations thereof. In some embodiments, the inflammatory mediator is a cytokine selected from interleukin-4, interleukin-5, interleukin-6, interleukin-9, interleukin-10, interleukin-13, interleukin-22, interleukin-25, interleukin-31, interleukin-33, interleukin-4 receptor, interleukin-5 receptor, interleukin-6 receptor, interleukin-9 receptor, interleukin-10 receptor, interleukin-13 receptor, interleukin-22 receptor, interleukin-25 receptor, interleukin-31 receptor, interleukin-33 receptor, thymic stromal lymphopoietin (TSLP), thymic stromal lymphopoietin receptor, tumor necrosis factor alpha, tumor necrosis factor alpha receptor, and combinations thereof.

In some embodiments, the allergic disorder comprises allergic inflammation and/or chronic inflammation. In some embodiments, the allergic disorder comprises allergic inflammation and is selected from allergic rhinitis, atopic dermatitis, allergic asthma, allergic conjunctivitis, gastro-intestinal inflammation, urticarial, latex allergy, and/or food allergy. In some embodiments, the allergic disorder is atopic dermatitis. In some embodiments, the subject has received and/or is currently receiving, cyclosporine, glucocorticoids, oclacitinib, by any route, or a combination thereof. In some embodiments, the allergic disorder is celiac disease, vasculitis, lupus, chronic obstructive pulmonary disease (COPD), irritable bowel disease (IBS), atherosclerosis, arthritis, systemic lupus erythematosus, multiple sclerosis, asthma, chronic peptic ulcer, sinusitis, tuberculosis, rheumatoid arthritis, periodontitis, ulcerative colitis, Crohn's disease, atopic dermatitis (eczema), rosacea, seborrheic dermatitis, and/or psoriasis.

In some embodiments, the biologic agent is a monoclonal antibody or a decoy receptor or a vaccine (e.g., a peptidic or recombinant DNA vaccine) aimed at inducing the production of an antibody or a decoy receptor. In some embodiments, the monoclonal antibody is one that targets the interleukin-4, interleukin-5, interleukin-6, interleukin-9, interleukin-10, interleukin-13, interleukin-22, interleukin-25, interleukin-31, interleukin-33, interleukin-4 receptor, interleukin-5 receptor, interleukin-6 receptor, interleukin-9 receptor, interleukin-10 receptor, interleukin-13 receptor, interleukin-22 receptor, interleukin-25 receptor, interleukin-31 receptor, interleukin-33 receptor, thymic stromal lymphopoietin (TSLP), thymic stromal lymphopoietin receptor, tumor necrosis factor alpha, tumor necrosis factor alpha receptor, and combinations thereof. In some embodiments, the monoclonal antibody is an anti-interleukin-31 receptor A antibody, an anti-interleukin-4 receptor alpha antibody, an anti-IL-13 antibody, an anti-IL-22 antibody, an anti-TSLP antibody, an anti-IL-31 antibody, or an anti-IL-33 antibody. In some embodiments, the monoclonal antibody is an anti-IL-33 antibody. In some embodiments, the monoclonal antibody is lokivetmab, nemolizumab, and/or dupilumab.

In some embodiments, the biologic agent is administered about every 1 week to about every 8 weeks. In some embodiments, the biologic agent is administered in combination with an anti-infectious agent. In some embodiments, the anti-infectious agent is an anti-septic, an antibiotic, an antifungal, or a combination thereof.

In some embodiments, the allergic disorder is atopic dermatitis, eczema, rosacea, seborrheic dermatitis, and/or psoriasis and the biologic is administered in combination with topical moisturizers and/or baths. In some embodiments, the composition comprises the biologic agent formulated with excipients, diluents, or combinations thereof. In some embodiments, the method provides reduced, delayed, and/or prevention of flares, relapses, and/or recurrences of lesions and/or itch in a subject with atopic dermatitis.

### BRIEF DESCRIPTION OF THE DRAWINGS

FIG. 1. Lokivetmab proactive therapy results.

FIG. 2. Comparison of duration of pruritus manifestations and skin lesion scores with or without lokivetmab proactive therapy in a dog model of atopic dermatitis. Although the lokivetmab abolished most of the pruritus manifestations, erythematous lesions still developed within 24 h of an epicutaneous allergen challenge.

FIG. 3. Flow chart of lokivetmab proactive therapy in client-owned dogs with atopic dermatitis (AD).

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 2nd Ed. (Cold Spring Harbor, N.Y., 1989); Ausubel et al. Current Protocols in Molecular Biology (Green Publishing Associates, Inc. and John Wiley & Sons, Inc., New York).

All publications, patent applications, patents, patent publications and other references cited herein are incorporated by reference in their entireties for the teachings relevant to the sentence and/or paragraph in which the reference is presented.

The present invention relates to methods for treating subjects that have had and/or are having and/or are at risk of having an allergic disorder by administering to the subject a biologic agent that targets inflammatory mediators before and/or during the early phase inflammation. In some embodiments, the biologic agent is not an anti-IgE antibody. In some embodiments, the biologic agent does not target IgE and in some embodiments, the biologic can target IgE, directly or indirectly. Typically an inflammatory component is present in allergic disorders, which can be acute or chronic in nature. The inflammatory component is due to the body's reaction towards an allergen triggering an inflammatory cascade. The inflammatory cascade is generally described as consisting of an early phase inflammatory reaction and a late phase inflammatory reaction. In some embodiments, a method of the present invention may reduce, delay, and/or prevent flares, symptoms, and/or relapses of the allergic disorder in the subject, optionally wherein the subject has minimal or no signs and/or symptoms of the allergic disorder.

In the early phase of the inflammatory reaction an IgE-mediated type I immediate hypersensitivity reaction occurs typically within minutes of allergen exposure. Reactions can be localized or can be systemic. In such reactions, IgE is bound to and IgE high affinity receptor (FcεRI) on mast cells and basophils crosslinked by allergen, resulting in the release of the cells' diverse preformed and newly synthesized inflammatory mediators (e.g., histamine, serglycin proteo-glycans, serine proteases, cytokines, tumor necrosis factor-alpha, leukotrienes, prostaglandins, and platelet-activating factor). Some of these released inflammatory mediators promote the local recruitment and activation of leukocytes, contributing to the development of late-phase reactions. The late phase of the inflammatory cascade is a reaction that typically develops after 2-6 h and peaks 6-9 h after allergen exposure. It is usually preceded by a clinically evident early-phase reaction and fully resolves in 1-2 days. Local recruitment and activation of T_{H}2 cells, eosinophils, basophils and other leukocytes, and persistent inflammatory mediator production by resident cells (such as mast cells) occur in the late phase which contributes to various long term symptoms associated with allergic inflammation.

The biologic agent of the invention administered to the subject may target inflammatory mediators before inflammation develops and/or when it begins during the early phase of inflammation, thereby interfering with the inflammatory cascade present in allergic disorders. For example, the biologic agent of the invention can be administered before inflammation develops or just when it begins in a subject. Modulation of the inflammatory cascade with the disclosed biologic agent does not only treat the primary systems of allergic disorders, such as systemic or localized inflammation, but more importantly also promotes reduced, delayed and/or prevention of relapses of inflammation and/or symptoms thereof. In some embodiments, the prevention is complete. Thus, with the continuous administration of the biologic agent alone or in combination with a primary treatment option for treating allergic disorders the subject should remain asymptomatic or paucisymptomatic for longer periods of time compared to treatment options that primarily focus on reducing the inflammatory response and symptoms thereof. The biologic agent of the invention can be any agent that is produced by means of biological processes involving recombinant DNA technology.

For example, in some embodiments, the biologic agent is a decoy receptor, which is a receptor that is able to recognize and bind specific growth factors or cytokines efficiently, but is not structurally able to signal or activate the intended receptor complex. Decoy receptors typically act as inhibitors, binding a ligand and keeping it from binding to its regular receptor, e.g., interleukin 1 receptor type II is a decoy receptor binding interleukin 1A and 1B to inhibit their binding to interleukin receptor 1 type I, deterring the inflammatory response which is generally promoted by the binding of type 1 interleukins to interleukin receptor 1 type I. Thus, in some embodiments, the biologic agent can be a decoy receptor for one or more of the inflammatory mediators of early phase inflammation.

In some embodiments, the biologic agent is a receptor construct (e.g., a fusion/chimeric protein), wherein a naturally occurring receptor is linked to an immunoglobulin frame. For example, the biologic agent can be a combination of a naturally occurring receptor that typically binds to one or more of the early phase inflammatory mediators coupled with an immunoglobulin such as an antibody designed to bind to one or more of the early phase inflammatory mediators present in the inflammatory cascade.

In some embodiments, the biologic agent is a monoclonal antibody that is “custom-designed” (using, e.g., hybridoma technology or other methods) to specifically target and/or bind to one or more early phase inflammatory mediators present in the inflammatory cascade. In some embodiments, the biologic agent is a “custom-designed” fragment of a monoclonal antibody.

In some embodiments, the biologic agent is a monoclonal antibody or a decoy receptor or a vaccine aimed at inducing the production of an antibody or a decoy receptor. Methods of preparation and use of antibodies and fragments thereof as biologic agents in the management of allergic disorders is discussed in more detail below.

The terms “a,” “an” and “the” are used herein to refer to one or to more than one (i.e., at least one) of the grammatical object of the article. By way of example, “an element” means at least one element and can include more than one element (e.g., a multiplicity or plurality of elements).

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, “one or more” can mean one, two, three, four, five, six, seven, eight, nine, ten or more, up to any number.

As used herein, the terms “subject” and “patient” are used interchangeably herein and refer to both human and nonhuman animals. A subject of this invention can be any subject that is susceptible to an allergic disorder and in particular embodiments; the subject of this invention is a human subject.

A “subject in need thereof” or “a subject in need of” is a subject known to have, or is suspected of having or developing and/or at risk of having or developing an allergic disorder.

An “appropriate therapy” for the treatment of an allergic disorder of this invention includes therapies well known in the art, including but not limited to, anti-inflammatory agents, immunomodulatory agents, and any combination thereof.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compounds of the invention for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

Also as used herein, the terms “treat,” “treating” or “treatment” refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, and/or change in clinical parameters of the condition, disorder, disease or illness, etc., as would be well known in the art.

Additionally as used herein, the terms “prevent,” “preventing” or “prevention” refer to any type of action that results in the absence, avoidance and/or delay of the onset and/or progression of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount” or “therapeutically effective amount” refers to an amount of a compound or composition of this invention that is sufficient to produce a desired effect, which can be a therapeutic and/or beneficial effect. The effective amount will vary with the age, general condition of the subject, the severity of the condition being treated, the particular agent administered, and the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

The term “biologically active” as used herein means an enzyme or protein having structural, regulatory, or biochemical functions of a naturally occurring molecule.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

Subjects with which the present invention is concerned include any subject susceptible to an allergic condition or disorder and are, in general, mammalian subjects, including humans, dogs, cats, and horses. The subjects may be of any gender, any ethnicity and any age.

The term “therapeutically effective amount” or “treatment effective amount” as used herein refers to the amount of a biologic agent determined to produce a therapeutic response in a subject. Such therapeutically effective amounts are readily ascertained by one of ordinary skill in the art.

The term “biologic agent” as used herein refers to any pharmaceutical drug product manufactured in, extracted from, or semi synthesized from biological sources. For example, they (or their precursors or components) can be isolated from living sources-human, animal, plant, fungal, or microbial or they can be specifically engineered macromolecular products like proteins-and nucleic acid-based drugs. Different from totally synthesized pharmaceuticals, they can include vaccines (which in some embodiments can be extracted directly from a biological source), blood, blood component, allergenics, somatic cells, gene therapies, tissues, recombinant therapeutic proteins, and/or living cells used in cell therapy. Biologic agents can be composed of sugars, proteins, and/or nucleic acids, and/or complex combinations of these substances, and/or may be living cells or tissues.

The term “antibody” as used herein refers to an intact immunoglobulin of any isotype, or a fragment thereof that can compete with the intact antibody for specific binding to the target antigen, and includes chimeric, humanized, caninized, equinized, felinized, fully human, fully canine, fully equine, fully feline, and bispecific antibodies. An intact antibody generally will comprise at least two full-length heavy chains and two full-length light chains, but in some instances may include fewer chains such as antibodies naturally occurring in camelids which may comprise only heavy chains. Antibodies according to the invention may be derived solely from a single source, or may be “chimeric,” that is, different portions of the antibody may be derived from two different antibodies. For example, the complementarity determining regions (CDRs) may be derived from a rat or murine source, while the framework region of the V region is derived from a different animal source, such as a human. The antibodies or binding fragments of the invention may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and mutins thereof, examples of which are described below.

The term “light chain” as used herein includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains according to the invention include kappa chains and lambda chains.

The term “heavy chain” includes a full-length heavy chain and fragments thereof having sufficient variable region sequence to confer binding specificity. A full-length heavy chain includes a variable region domain, V_{II}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the —COOH end. Heavy chains according to the invention may be of any isotype, including IgG (including IgG1, IgG2, IgG3 and IgG4 subtypes), IgA (including IgA₁ and IgA₂ subtypes), and IgM but not IgE.

The term “immunologically functional fragment” (or simply “fragment”) of an immunoglobulin chain, as used herein, refers to a portion of an antibody light chain or heavy chain that lacks at least some of the amino acids present in a full-length chain but which is capable of binding specifically to an antigen. Such fragments are biologically active in that they bind specifically to the target antigen and can compete with intact antibodies for specific binding to a given epitope. In one aspect of the invention, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of intact antibodies. Immunologically functional immunoglobulin fragments of the invention include, but are not limited to, Fab, Fab′, F(ab′)₂, Fv, domain antibodies and single-chain antibodies, and may be derived from any mammalian source, including but not limited to human, mouse, rat, camelid or rabbit. It is contemplated further that a functional portion of the inventive antibodies, for example, one or more CDRs, could be covalently bound to a second protein or to a small molecule to create a therapeutic agent directed to a particular target in the body, possessing bifunctional therapeutic properties, or having a prolonged serum half-life.

The term “Fab fragment” as used herein is comprised of one light chain and the CHI and variable regions of one heavy chain. The heavy chain of a Fab molecule cannot form a disulfide bond with another heavy chain molecule.

The term “Fc” region as used herein contains two heavy chain fragments comprising the C.sub.H1 and C.sub.H2 domains of an antibody. The two heavy chain fragments are held together by two or more disulfide bonds and by hydrophobic interactions of the CH3 domains.

The term “Fab′ fragment” as used herein contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form a F(ab′)₂ molecule.

The term “F(ab′)₂ fragment” as used herein contains two light chains and two heavy chains containing a portion of the constant region between the C_{H1} and C_{H2} domains, such that an interchain disulfide bond is formed between the two heavy chains. A F(ab′)2 fragment thus is composed of two Fab′ fragments that are held together by a disulfide bond between the two heavy chains.

The term “Fv region” as used herein comprises the variable regions from both the heavy and light chains, but lacks the constant regions.

The term “single-chain antibodies” as used herein are Fv molecules (scFv) in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference.

The term “domain antibody” as used herein is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “bivalent antibody” as used herein comprises two antigen binding sites. In some instances, the two binding sites have the same antigen specificities. However, bivalent antibodies may be bispecific (see below).

The term “multispecific antibody” as used herein is one that targets more than one antigen or epitope.

The term “bispecific,” “dual-specific” or “bifunctional” antibody as used herein is a hybrid antibody having two different antigen binding sites. Bispecific antibodies are a species of multispecific antibody and may be produced by a variety of methods including, but not limited to, fusion of hybridomas or linking of Fab′ fragments. See, e.g., Songsivilai & Lachmann (1990), Clin. Exp. Immunol. 79:315-321; Kostelny et al. (1992), J. Immunol. 148:1547-1553. The two binding sites of a bispecific antibody will bind to two different epitopes, which may reside on the same or different protein targets.

As discussed herein, a variety of selective biologic agents useful for regulating the activity of inflammatory mediators present during early phase inflammation are provided. In some embodiments, IgE is included as a target and in some embodiments, IgE is not included as a target for an early phase inflammatory mediator. These biologic agents include, for instance, antibodies and immunologically functional fragments thereof that contain an antigen binding domain (e.g., single chain antibodies, domain antibodies, immunoadhesions, and polypeptides with an antigen binding region) and specifically bind to the targeted inflammatory mediator(s). Exemplary targeted inflammatory mediators include, but are not limited to histamines, leukotrienes, prostaglandins, cytokines, platelet activating factor (PAF), growth factors, an immunoglobulin (which may or may not include IgE), and combinations thereof. In some embodiments, cytokines are selected as a targeted early phase inflammatory mediator. For example, cytokines selected from interleukin-4, interleukin-5, interleukin-6, interleukin-9, interleukin-10, interleukin-13, interleukin-22, interleukin-25, interleukin-31, interleukin-33, interleukin-4 receptor, interleukin-5 receptor, interleukin-6 receptor, interleukin-9 receptor, interleukin-10 receptor, interleukin-13 receptor, interleukin-22 receptor, interleukin-25 receptor, interleukin-31 receptor, interleukin-33 receptor, thymic stromal lymphopoietin (TSLP), thymic stromal lymphopoietin receptor, tumor necrosis factor alpha, tumor necrosis factor alpha receptor, and combinations thereof.

Exemplary biologic agents include anti-interleukin-31 receptor A antibodies, anti-interleukin-4 receptor alpha antibodies, and/or anti-interleukin-31 antibodies. In some embodiments, a biologic agent (e.g., antibody) targets early phase inflammatory mediators comprising an anti-interleukin-4, anti-interleukin-5, anti-interleukin-6, anti-interleukin-9, anti-interleukin-10, anti-interleukin-13, anti-interleukin-22, anti-interleukin-25, anti-interleukin-31, anti-interleukin-33, anti-interleukin-4 receptor, anti-interleukin-5 receptor, anti-interleukin-6 receptor, anti-interleukin-9 receptor, anti-interleukin-10 receptor, anti-interleukin-13 receptor, anti-interleukin-22 receptor, anti-interleukin-25 receptor, anti-interleukin-31 receptor, anti-interleukin-33 receptor, anti-thymic stromal lymphopoietin (TSLP) receptor, anti-thymic stromal lymphopoietin receptor, anti-tumor necrosis factor alpha receptor, and combinations thereof. In some embodiments, a biologic agent (e.g., antibody) targets early phase inflammatory mediators comprising an anti-interleukin-31 receptor A antibody, an anti-interleukin-4 receptor alpha antibody, an anti-IL-13 antibody, an anti-IL-22 antibody, an anti-TSLP antibody, an anti-IL-31 antibody, and/or an anti-IL-33 antibody. Exemplary antibodies include, but are not limited to, lokivetmab, nemolizumab, and/or duplimab.

Variable Domains of Antibodies. Also provided are antibodies that comprise a light chain variable region as described herein, and/or a heavy chain variable region as described herein

CDRs of Antibodies. The antibodies and immunological functional fragments that are provided can include one or more CDRs (e.g., one, two, three, four, five or all six CDRs). The heavy and light chain variable regions and the CDRs that are disclosed herein can be used to prepare any of the various types of immunologically functional fragments that are known in the art including, but not limited to, domain antibodies, Fab fragments, Fab′ fragments, F(ab′)₂ fragments, Fv fragments, single-chain antibodies, sdFvs, scFvs, etc.

Single-chain Variable Fragments. Single chain variable fragment (scFv) antibodies can be produced in accordance with known techniques or variations thereof that will be apparent to those skilled in the art. See generally U.S. Pat. No. 4,946,778 to Ladner et al. and U.S. Pat. No. 5,258,498 to Huston and Opperman; see also U.S. Pat. Nos. 8,097,704; 8,043,830; 7,943,144; 7,910,702; and 7,816,334.

Bispecific or Bifunctional Antibodies. The antibodies that are provided also include bispecific and bifunctional antibodies that include one or more CDRs or one or more variable regions as described above. A bispecific or bifunctional antibody in some instances is an artificial hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann, 1990, Clin. Exp. Immunol. 79: 315-321; Kostelny et al., 1992, J. Immunol. 148: 1547-1553.

Chimeric, Humanized, Caninized, Equinized, and Felinized Antibodies. Chimeric and humanized antibodies are also provided. Monoclonal antibodies for use as therapeutic agents may be modified in various ways prior to use. One example is a “chimeric” antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., Proc. Natl. Acad. Sci. USA 81:6851-6855 (1985), which are hereby incorporated herein by reference. CDR grafting is described, for example, in U.S. Pat. Nos. 6,180,370, 5,693,762, 5,693,761, 5,585,089, and 5,530,101, which are all hereby incorporated by reference for all purposes.

Generally, the goal of making a chimeric antibody is to create a chimera in which the number of amino acids from the intended subject species is maximized. One example is the “CDR-grafted” antibody, in which the antibody comprises one or more complementarity determining regions (CDRs) from a particular species or belonging to a particular antibody class or subclass, while the remainder of the antibody chain(s) is/are identical with or homologous to a corresponding sequence in antibodies derived from another species or belonging to another antibody class or subclass. For use in humans, the V region or selected CDRs from a rodent antibody often are grafted into a human antibody, replacing the naturally-occurring V regions or CDRs of the human antibody.

One useful type of chimeric antibody is a “humanized” antibody. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089, and 5,693,762; Jones et al., 1986, Nature 321:522-25; Riechmann et al., 1988, Nature 332:323-27; Verhoeyen et al., 1988, Science 239:1534-36).

Caninized, equinized, and felinized antibodies are known, and are made in like manner as described in connection with humanized antibodies above. See, e.g., U.S. Pat. Nos. 8,076,456; 7,261,890; 6,881,557; 6,504,013; 5,760,185; and US Patent Application Pub. No. US 2010/0061988.

In one aspect of the invention, the CDRs of the light and heavy chain variable regions of the antibodies provided herein are grafted to framework regions (FRs) from antibodies from the same, or a different, phylogenetic species. For example, the CDRs of the light and heavy chain variable regions of the antibody can be grafted to consensus human, canine, equine, or feline FRs. To create consensus FRs, FRs from several heavy chain or light chain amino acid sequences of the desired species may be aligned to identify a consensus amino acid sequence. In other embodiments, the FRs of the antibody heavy chain or light chain are replaced with the FRs from a different heavy chain or light chain.

In one aspect of the invention, rare amino acids in the FRs of the heavy and light chains of the biologic agent (e.g., antibody or fragment thereof) are not replaced, while the rest of the FR amino acids are replaced. A “rare amino acid” is a specific amino acid that is in a position in which this particular amino acid is not usually found in an FR. Alternatively, the grafted variable regions from the antibody may be used with a constant region that is different from the constant region of. In other embodiments of the invention, the grafted variable regions are part of a single chain Fv antibody.

Nucleic acid molecules that encode a biologic of this invention, one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing, are also provided herein. The nucleic acid molecules can be any length, and/or can comprise one or more additional sequences, for example, regulatory sequences, and/or be part of a larger nucleic acid molecule, for example, a vector. The nucleic acid molecules can be single-stranded or double-stranded and can comprise RNA and/or DNA nucleotides, and artificial variants thereof (e.g., peptide nucleic acids). In some embodiments, the nucleic acid molecules can be present in a composition comprising a pharmaceutically acceptable carrier and can be used in the methods of this invention for therapeutic applications.

In another aspect, the present invention provides vectors comprising a nucleic acid encoding a polypeptide of the invention or a portion thereof (e.g., a fragment containing one or more CDRs or one or more variable region domains). Examples of vectors include, but are not limited to, plasmids, viral vectors, non-episomal mammalian vectors and expression vectors, for example, recombinant expression vectors, Tumor-inducing (Ti) plasmids, ballistic particles carrying recombinant nucleic acids, etc. The recombinant expression vectors of the invention can comprise a nucleic acid of the invention in a form suitable for expression of the nucleic acid in a host cell. The recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells (e.g., SV40 early gene enhancer, Rous sarcoma virus promoter and cytomegalovirus promoter), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237, incorporated by reference herein in their entireties), and those that direct inducible expression of a nucleotide sequence in response to particular treatment or condition (e.g., the metallothionin promoter in mammalian cells and the tet-responsive and/or streptomycin responsive promoter in both prokaryotic and eukaryotic systems. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

In another aspect, the present invention provides host cells into which a recombinant expression vector of the invention has been introduced. A host cell can be any prokaryotic cell (for example, E. coli) or eukaryotic cell (for example, yeast, insect, plant, or mammalian cells (e.g., CHO cells)). Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die), among other methods.

Single chain antibodies of the present invention may be formed by linking heavy and light chain variable domain (Fv region) fragments via an amino acid bridge (short peptide linker), resulting in a single polypeptide chain. Such single-chain Fvs (scFvs) may be prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains. By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

Antibodies provided herein that are of one subclass can be changed to antibodies from a different subclass using subclass switching methods. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See, e.g., Lantto et al., 2002, Methods Mol. Biol. 178:303-16.

Moreover, techniques for deriving antibodies having different properties (i.e., varying affinities for the antigen to which they bind) are also known. One such technique, referred to as chain shuffling, involves displaying immunoglobulin variable domain gene repertoires on the surface of filamentous bacteriophage, often referred to as phage display. Chain shuffling has been used to prepare high affinity antibodies to the hapten 2-phenyloxazol-5-one, as described by Marks et al., 1992, BioTechnology, 10:779.

Conservative modifications may be made to the heavy and light chains described herein (and corresponding modifications to the encoding nucleic acids) to produce a biologic agent (e.g., antibody or fragment thereof) having functional and biochemical characteristics. Methods for achieving such modifications are described herein.

Antibodies and functional fragments thereof according to the invention may be further modified in various ways. For example, if they are to be used for therapeutic purposes, they may be conjugated with polyethylene glycol (pegylated) to prolong the serum half-life or to enhance protein delivery. Alternatively, the V region of the subject antibodies or fragments thereof may be fused with the Fc region of a different antibody molecule. The Fc region used for this purpose may be modified so that it does not bind complement, thus reducing the likelihood of inducing cell lysis in the patient when the fusion protein is used as a therapeutic agent. In addition, the subject antibodies or functional fragments thereof may be conjugated with human serum albumin to enhance the serum half-life of the antibody or fragment thereof. Another useful fusion partner for the inventive antibodies or fragments thereof is transthyretin (TTR). TTR has the capacity to form a tetramer, thus an antibody-TTR fusion protein can form a multivalent antibody which may increase its binding avidity.

Alternatively, substantial modifications in the functional and/or biochemical characteristics of the antibodies and fragments described herein may be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain. A “conservative amino acid substitution” may involve a substitution of a native amino acid residue with a normative residue that has little or no effect on the polarity or charge of the amino acid residue at that position. Furthermore, any native residue in the polypeptide may also be substituted with alanine, as has been previously described for alanine scanning mutagenesis.

Amino acid substitutions (whether conservative or non-conservative) of the subject antibodies can be implemented by those skilled in the art by applying routine techniques. Amino acid substitutions can be used to identify important residues of the antibodies provided herein, or to increase or decrease the affinity of these antibodies for human IgE or for modifying the binding affinity of other biologic agents described herein.

A biologic agent of the present invention (e.g., antibody or fragment thereof) may be prepared by any of a number of conventional techniques. For example, antibodies or fragments thereof may be produced by recombinant expression systems, using any technique known in the art. See, for example, Monoclonal Antibodies, Hybridomas: A New Dimension in Biological Analyses, Kennet et al. (eds.) Plenum Press, New York (1980): and Antibodies: A Laboratory Manual, Harlow and Lane (eds.), Cold Spring Harbor Laboratory Press, Cold Spring Harbor, N.Y. (1988).

Antibodies of the present invention and binding fragments thereof can be produced in hybridoma cell lines or in cell lines other than hybridomas. Expression constructs encoding the antibodies can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216, 4,912,040, 4,740,461, and 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs of the invention typically comprise a nucleic acid molecule encoding a polypeptide comprising one or more of the following: a heavy chain constant region; a heavy chain variable region; a light chain constant region; a light chain variable region; one or more CDRs of the light or heavy chain of the antibody targeting the early phase inflammatory mediator. These nucleic acid sequences are inserted into an appropriate expression vector using standard ligation techniques. In one embodiment, the canine, equine, feline, or human antibody heavy or light chain constant region is appended to the C-terminus of the heavy or light chain variable region and is ligated into an expression vector. The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, that is, an oligonucleotide molecule located at the 5′ or 3′ end of the coding sequence, the oligonucleotide sequence encoding polyHis, or another “tag” for which commercially available antibodies exist, such as FLAG®™, HA (hemagglutinin from influenza virus), or myc. The tag is typically fused to the antibody protein upon expression, and can serve as a means for affinity purification of the antibody from the host cell. Affinity purification can be accomplished, for example, by column chromatography using antibodies against the tag as an affinity matrix. Optionally, the tag can subsequently be removed from the purified antibody polypeptide by various means such as using certain peptidases for cleavage.

Flanking sequences in the expression vector may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with a suitable oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, column chromatography, or other methods known to the skilled artisan. The selection of suitable enzymes to accomplish this purpose will be readily apparent to those skilled in the art.

An origin of replication is typically a part of prokaryotic expression vectors, particularly those purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 is suitable for most gram-negative bacteria and various origins (e.g., SV40, polyoma, adenovirus, vesicular stomatitis virus (VSV), or papillomaviruses such as HPV or BPV) are useful for cloning vectors in mammalian cells. Generally, a mammalian origin of replication is not needed for mammalian expression vectors (for example, the SV40 origin is often used only because it contains the early promoter).

The expression and cloning vectors of the present invention will typically contain a promoter that is recognized by the host organism and operably linked to nucleic acid encoding the biologic agent (e.g., antibody or fragment thereof). Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continuous gene product production; that is, there is little or no experimental control over gene expression. A large number of promoters, recognized by a variety of potential host cells, are well known. A suitable promoter is operably linked to the DNA encoding a biologic agent (e.g., antibody or fragment thereof) by removing the promoter from the source DNA by restriction enzyme digestion or amplifying the promoter by polymerase chain reaction and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Particular promoters useful in the practice of the recombinant expression vectors of the invention include, but are not limited to: the SV40 early promoter region; the CMV promoter; the promoter contained in the 3′ long terminal repeat of Rous sarcoma virus; the herpes thymidine kinase promoter; the regulatory sequences of the metallothionine; prokaryotic expression vectors such as the beta-lactamase promoter; or the tac promoter. Also available for use are the following animal transcriptional control regions, which exhibit tissue specificity and have been utilized in transgenic animals: the elastase I gene control region that is active in pancreatic acinar cells; the insulin gene control region that is active in pancreatic beta cells; the mouse mammary tumor virus control region that is active in testicular, breast, lymphoid and mast cells; the albumin gene control region that is active in; the alpha-feto-protein gene control region that is active in liver; the alpha 1-antitrypsin gene control region that is active in the liver; the beta-globin gene control region that is active in myeloid cells; the myelin basic protein gene control region that is active in oligodendrocyte cells in the brain; the myosin light chain-2 gene control region that is active in skeletal muscle; the gonadotropic releasing hormone gene control region that is active in the hypothalamus; and the immunoglobulin gene control region that is active in lymphoid.

An enhancer sequence may be inserted into the vector to increase the transcription in higher eukaryotes of a nucleic acid encoding an antibody or fragments thereof of the present invention. Enhancers are cis-acting elements of DNA, usually about 10-300 base pairs in length, that act on promoters to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). An enhancer sequence from a virus also can be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to a nucleic acid molecule, it is typically placed at a site 5′ to the promoter.

In expression vectors, a transcription termination sequence is typically located 3′ of the end of a polypeptide-coding region and serves to terminate transcription. A transcription termination sequence used for expression in prokaryotic cells typically is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes used in expression vectors encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex media. Examples of selectable markers include the kanamycin resistance gene, the ampicillin resistance gene and the tetracycline resistance gene. A bacterial neomycin resistance gene can also be used for selection in both prokaryotic and eukaryotic host cells.

Other selection genes can be used to amplify the gene that will be expressed. Amplification is a process whereby genes that cannot in single copy be expressed at high enough levels to permit survival and growth of cells under certain selection conditions are reiterated in tandem within the chromosomes of successive generations of recombinant cells. Examples of suitable amplifiable selectable markers for mammalian cells include dihydrofolate reductase (DHFR) and promoterless thymidine kinase. In the use of these markers mammalian cell transformants are placed under selection pressure wherein only the transformants are uniquely adapted to survive by virtue of the selection gene present in the vector. Selection pressure is imposed by culturing the transformed cells under conditions in which the concentration of selection agent in the medium is successively increased, thereby permitting survival of only those cells in which the selection gene has been amplified. Under these circumstances, DNA adjacent to the selection gene, such as DNA encoding an antibody of the invention, is co-amplified with the selection gene. As a result, increased quantities of biologic agent (i.e., antibody) are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, for example where glycosylation is desired in a eukaryotic host cell expression system, various presequences can be manipulated to improve glycosylation or yield. For example, the peptidase cleavage site of a particular signal peptide can be altered, or pro-sequences added, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated yet active form of the desired polypeptide, if the enzyme cuts at such area within the mature polypeptide.

Where a commercially available expression vector lacks some of the desired flanking sequences as described above, the vector can be modified by individually ligating these sequences into the vector. After the vector has been chosen and modified as desired, a nucleic acid molecule encoding a biologic agent (e.g., antibody) is inserted into the proper site of the vector.

The completed vector containing sequences encoding the inventive antibody or immunologically functional fragment thereof or other biologic of this invention is inserted into a suitable host cell for amplification and/or polypeptide production. The transformation of an expression vector for a biologic agent (e.g., antibody) into a selected host cell may be accomplished by well-known methods including methods such as transfection, infection, calcium chloride, electroporation, microinjection, lipofection, DEAE-dextran method, or other known techniques. The method selected will in part be a function of the type of host cell to be used. These methods and other suitable methods are well known to the skilled artisan.

The transformed host cell, when cultured under appropriate conditions, synthesizes a biologic agent (e.g., an antibody) that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (ATCC), such as Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, the best cell line for expressing a particular DNA construct may be selected by testing various cell lines to determine which ones have the highest levels of expression and produce antibodies with desired binding properties to its corresponding target.

In addition to the foregoing, systems for the production of transgenic plants that produce antibodies (e.g., plantibodies), and from which the antibodies are collected, are known and can also be used to carry out the present invention. Examples of such plants, methods of making such plants, and methods of using such plants for the production and collection of antibodies are described in, for example, U.S. Pat. Nos. 8,071,333; 7,781,647; 7,736,648; 7,247,711; 6,852,319 6,841,659; 6,040,498; and 5,959,177.

In certain embodiments, the invention also provides compositions comprising a subject biologic agent (e.g., antibody or fragment thereof) together with one or more of the following: a pharmaceutically acceptable diluent; an excipient; a carrier; a solubilizer; an emulsifier; a preservative; and/or an adjuvant. Such compositions may contain an effective amount of the biologic agent. Thus, use of the biologic agent is provided herein in the preparation of a pharmaceutical composition or medicament is also included. Such compositions can be used in the prevention and/or treatment of signs of a variety of allergic disorders such as listed below in the section on exemplary utilities.

Acceptable formulation components for pharmaceutical preparations are nontoxic to recipients at the dosages and concentrations employed. In addition to the biologic agent provided, compositions according to the invention may contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as Mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. Suitable vehicles or carriers for such compositions include water for injection, physiological saline solution or artificial cerebrospinal fluid, possibly supplemented with other materials common in compositions for parenteral administration. Neutral buffered saline or saline mixed with serum albumin are further exemplary vehicles.

Compositions comprising a biologic agent of this invention may be prepared for storage by mixing the selected composition having the desired degree of purity with optional formulation agents in the form of a lyophilized cake or an aqueous solution. Further, the biologic agent may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

A pharmaceutical composition may involve an effective quantity of a biologic agent in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert materials, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions are in the form of sustained- or controlled-delivery formulations. Techniques for formulating a variety of other sustained- or controlled-delivery means, such as liposome carriers, bio-erodible microparticles or porous beads and depot injections can be. Sustained-release preparations may include semipermeable polymer matrices in the form of shaped articles, e.g. films, or microcapsules, polyesters, hydrogels, polylactides, copolymers of L-glutamic acid and gamma ethyl-L-glutamate, poly (2-hydroxyethyl-methacrylate), ethylene vinyl acetate or poly-D(−)-3-hydroxybutyric acid. Sustained release compositions may also include liposomes, which can be prepared by any of several methods known in the art.

The pharmaceutical composition to be used for in vivo administration typically is sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The composition may be formulated for transdermal delivery, optionally with the inclusion of microneedles, microprojectiles, patches, electrodes, adhesives, backings, and/or packaging, or formulations for jet delivery, in accordance with known techniques. See, e.g., U.S. Pat. Nos. 8,043,250; 8,041,421; 8,036,738; 8,025,898; 8,017,146.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The present invention provides kits for producing multi-dose or single-dose administration units. For example, kits according to the invention may each contain both a first container having a dried biologic and a second container having an aqueous diluent, including for example single and multi-chambered pre-filled syringes (e.g., liquid syringes, lyosyringes or needle-free syringes).

The pharmaceutical compositions of the invention can be delivered parenterally, typically by injection. Injections can be intraocular, intraperitoneal, intraportal, intramuscular, intravenous, intrathecal, intracerebral (intra-parenchymal), intracerebroventricular, intraarterial, intralesional, perilesional or subcutaneous. Eye drops can be used for intraocular administration. In some instances, injections may be localized to the vicinity of a particular bone or bones to which the treatment is targeted. For parenteral administration, the biologic agent may be administered in a pyrogen-free, parenterally acceptable aqueous solution comprising the biologic agent in a pharmaceutically acceptable vehicle. A particularly suitable vehicle for parenteral injection is sterile distilled water in which the biologic agents are formulated as a sterile, isotonic solution, properly preserved.

Pharmaceutical compositions comprising the subject biologic agent may be administered by bolus injection or continuously by infusion, by implantation device, sustained release systems or other means for accomplishing prolonged release. The pharmaceutical composition also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous release. The preparation may be formulated with agent, such as injectable microspheres, bio-erodible particles, polymeric compounds (such as polylactic acid; polyglycolic acid; or copoly (lactic/glycolic) acid (PLGA), beads or liposomes, that can provide controlled or sustained release of the product which may then be delivered via a depot injection. Formulation with hyaluronic acid has the effect of promoting sustained duration in the circulation.

The subject compositions comprising a biologic agent may be formulated for inhalation. In these embodiments, a biologic agent is formulated as a dry powder for inhalation, or biologic agent inhalation solutions may also be formulated with a propellant for aerosol delivery, such as by nebulization.

Certain pharmaceutical compositions of the invention can be delivered through the digestive tract, such as orally. The subject biologic agent that is administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized. Additional agents can be included to facilitate absorption of the biologic agent. For oral administration, modified amino acids may be used to confer resistance to digestive enzymes. Diluents, flavorings, low melting point waxes, vegetable oils, lubricants, suspending agents, tablet disintegrating agents, and binders may also be employed.

The subject compositions comprising a biologic agent also may be used ex vivo. In such instances, cells, tissues or organs that have been removed from the patient are exposed to or cultured with the biologic agent. The cultured cells may then be implanted back into the patient or a different patient or used for other purposes.

In certain embodiments, the biologic agent can be delivered by implanting certain cells that have been genetically engineered, using methods such as those described herein, to express and secrete the polypeptide. Such cells may be animal or human cells, and may be autologous, heterologous, or xenogenic, or may be immortalized. In order to decrease the chance of an immunological response, the cells may be encapsulated to avoid infiltration of surrounding tissues. Encapsulation materials are typically biocompatible, semi-permeable polymeric enclosures or membranes that allow the release of the protein product(s) but prevent the destruction of the cells by the patient's immune system or by other detrimental factors from the surrounding tissues.

Subjects to be treated by a method and/or composition of the present invention include any subject afflicted with a disorder or condition in which reduction of inflammatory mediators associated with early phase inflammation, not including IgE, would be beneficial. The subject receiving treatment can be a canine, feline, equine, or human. In particular, the subject can be a human, dog, cat, or horse.

In some embodiments, the subject is asymptomatic or paucisymptomatic with or without currently receiving an anti-inflammatory agent, an anti-allergy agent, and immunomodulatory agent, or a combination thereof. The term “asymptomatic” refers to a subject who is a carrier for a disease but experiences no symptoms (e.g., a condition might be asymptomatic if it fails to show the noticeable symptoms with which it is usually associated with) whereas “paucisymptomatic” describes subjects which are almost clear of symptoms and/or with “clear” or “almost clear” clinical signs. Examples of anti-allergy agents and immunomodulatory agents include, but should not be limited to, glucocorticoids, non-steroidal anti-inflammatory agents, leukotriene antagonists, Janus kinase (JAK) inhibitors, immunoglobulins, anti-histamines, allergen-specific or monospecific immunotherapy, and combinations thereof.

Allergic disorders to be treated with the compositions of the invention comprise allergic inflammation and/or chronic inflammation. Examples of chronic inflammation include, but are not limited to, celiac disease, vasculitis, lupus, chronic obstructive pulmonary disease (COPD), irritable bowel disease (IBS), atherosclerosis, arthritis, systemic lupus erythematosus, multiple sclerosis, asthma, chronic peptic ulcer, sinusitis, tuberculosis, rheumatoid arthritis, periodontitis, ulcerative colitis, Crohn's disease, atopic dermatitis (eczema), rosacea, seborrheic dermatitis, and/or psoriasis. Examples of allergic inflammation include, but are not limited to, allergic rhinitis, atopic dermatitis, allergic asthma, allergic conjunctivitis, gastro-intestinal inflammation, urticarial, latex allergy, and/or food allergy.

In some embodiments, the allergic disorder to be treated is an inflammatory skin disorder. Examples include, but should not be limited to, atopic dermatitis, eczema, rosacea, seborrheic dermatitis, and/or psoriasis. In some embodiments, the subject to be treated has received and/or is currently receiving treatment, such as cyclosporine, glucocorticoids, oclacitinib, topical moisturizers, baths, or combinations thereof. In some embodiments, the inflammatory skin disorder is atopic dermatitis and the subject being treated is a dog or a cat.

A pharmaceutical composition of the present invention may be administered to prevent, reduce, and/or inhibit an allergic disorder in a subject in need thereof. An “effective amount” refers generally to an amount that is a sufficient, but non-toxic, amount of the active ingredient (i.e., the biologic agent) to achieve the desired effect, which is a reduction or elimination in the severity and/or frequency of symptoms and/or improvement or remediation of damage (e.g., a reduction, delay, and/or prevention of flares (e.g., relapses) and/or recurrences of lesions and/or itch in a subject with atopic dermatitis). A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom (e.g., a flare).

In general, toxicity and therapeutic efficacy of the biologic agent (e.g., antibody or fragment) can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from cell culture and/or animal studies can be used in formulating a range of dosages for subjects for treatment. The dosage of the active ingredient typically lines within a range of circulating concentrations that include the ED₅₀ with little or no toxicity. The dosage can vary within this range depending upon the dosage form employed and the route of administration utilized.

The effective amount of a pharmaceutical composition comprising a biologic agent (e.g., antibody or fragment thereof) to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the biologic agent (e.g., antibody or fragment thereof) is being used, the route of administration, and the size (body weight, body surface or organ size) and/or condition (the age and general health) of the subject. Clinicians may titer the dosage and modify the route of administration to obtain the optimal therapeutic effect. Typical dosages range from about 1.0 μg/Kg to about 500 mg/Kg body weight (e.g., from about 1.0 μg/Kg to about 100 μg/Kg, or from about 500 μg/Kg to about 1 mg/Kg, or from about 5 mg/Kg to about 100 mg/Kg subject body weight), or more.

The dosing frequency will depend upon the pharmacokinetic parameters of the biologic agent in the formulation. For example, a clinician will administer the composition until a dosage is reached that achieves the desired effect. The composition may therefore be administered as a single dose or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

In some embodiments, the biologic agent is administered in combination with another therapeutic agent. Examples of therapeutic agents include, but are not limited to, anti-infectious agent (e.g., anti-septic agent, anti-biotic agent, anti-fungal agent), an anti-allergy agent, and/or an immunomodulatory agent. The therapeutic agent can be administered simultaneously with the biologic agent and/or can be administered at a different time point. The route of administration of the therapeutic agent can be the same or different as the route of administration for the biologic agent. In some embodiments, the biologic agent is administered in combination with topical moisturizers and/or baths.

To treat an allergic disorder characterized by abnormal or excess amount of early phase inflammatory mediators, a composition comprising the biologic agent may be administered to a subject in an amount and for a time sufficient to induce a sustained improvement in at least one indicator that reflects the severity of the allergic disorder. For example, the biologic agent can be administered about every 1, 2, 3, 4, 5, 6, 7, 8, 9, or 10 or more weeks. In other embodiments, the biologic agent is administered about 1, 2, 3, 4, 5, 6, or 7 or more times a week. An improvement is considered “sustained” if the subject exhibits the improvement on at least two occasions separated by at least one to seven days, or in some instances one to six weeks. The appropriate interval will depend to some extent on what allergic disorder is being treated; it is within the purview of those skilled in the art to determine the appropriate interval for determining whether the improvement is sustained. For example, improvement is considered sustained if a subject having atopic dermatitis exhibits a reduction in the number of and/or a delay in flares, relapses and/or recurrences of lesions and/or itch.

According to some embodiments of the present invention a kit is provided. A kit may include one or more biologic agent(s) and/or a pharmaceutical composition as described herein. Some kits include such biologic agent(s) and/or composition(s) in a container (e.g., vial or ampule), and may also include instructions for use of said biologic agent(s) and/or composition(s) in the various methods disclosed above. The biologic agent and/or composition can be in various forms, including, for instance, as part of a solution or as a solid (e.g., lyophilized powder). The instructions may include a description of how to prepare (e.g., dissolve or resuspend) the biologic agent in an appropriate fluid and/or how to administer the biologic agent for the treatment of the allergic disorder described.

The kits may also include various other components, such as buffers, salts, complexing metal ions and other agents described above in the section on pharmaceutical compositions. These components may be included with the biologic agent or may be in separate containers. The kits may also include other therapeutic agents for administration with the biologic agent. Examples of such agents include, but are not limited to, agents to treat the allergic disorders described above.

The following examples are provided solely to illustrate certain aspects of the antibodies, fragments and compositions that are provided herein and thus should not be construed to limit the scope of the claimed invention.

### EXAMPLES

### Example 1

### Phase 1: Experimental Acute Canine AD Model.

We used four Maltese-beagle atopic dogs that had been sensitized to the HDM Dermatophagoides farinae, as described previously (Olivry T, Wofford J, Paps J S et al. Stratum corneum removal facilitates experimental sensitization to allergens in atopic dogs. Vet Dermatol 2011; 22: 188-196). There were three males and one female ranging from 3- to 8-year-old. All dogs were injected once subcutaneously with the lokivetmab at an average dosage of 2.75 mg/kg (range: 2.5-3.0 mg/kg); this dosage, higher than that approved in the USA, was selected in an attempt to not miss the detection of any preventive effect on lesions or itch. Seven days after the mAb injection, the dogs were challenged epicutaneously with 25 mg of lyophilized HDM (Greer Laboratories; Lenoir, N.C., USA) in 1 mL of mineral oil. We applied this suspension to an area of c.200 cm2 on the previously clipped right side of the abdomen, as reported previously (Paps J S, Baumer W, Olivry T. Development of an allergen-induced atopic itch model in dogs: a preliminary report. Acta Derm Venereol 2016; 96: 400-401).

Starting 24 h before HDM challenge and continuing for 24 h after it, dogs were video-monitored and the duration of pruritus manifestations (DPM), such as scratching, biting, licking or chewing on the right abdomen, was assessed as reported previously. The DPM after the challenge was subtracted from the value for 24 h before it to give the DPM associated with the sole allergen provocation (i.e. the “post-minus-pre-” number of seconds in the first 24 h after challenge). Immediately before and 24 h post-HDM challenge, erythematous macules, oedema, papules/pustules and excoriations were scored as 0 (absent), 1 (faint, mild), 2 (moderate) or 3 (strong, severe). The grades for each lesion were added to yield a skin lesion score (SLS) with an achievable maximum of 12. To serve as a “no intervention” (i.e. untreated) control), we used the median DPM and SLS values obtained during all HDM challenges in the preceding three years with the same dogs. Statistical analyses were not performed due to the small number of dogs enrolled in this phase.

Phase 2: Prospective Study of Client-Owned Dogs with AD

This study was a prospective, uncontrolled, open-label trial with client-owned atopic dogs treated proactively with monthly-to-bimonthly lokivetmab injections. All dogs with atopic dermatitis (AD) that received the first lokivetmab injection at the NC State Veterinary Hospital dermatology service between 1 February 2016 and 30 March 2017 were eligible to enter the study. Owners consented to the injections of lokivetmab per standard recommendations and to the need of reporting any flare needing anti-allergic treatment escalation. The dogs with AD were treated with their standard-of-care medications until their disease was deemed to be controlled. All dogs were then prescribed injections of the anti-interleukin-31 monoclonal antibody lokivetmab (Cytopoint, Zoetis), at the targeted dosage of 1 to 2 mg/kg every 4 weeks (lower dosage) to 4-8 weeks (higher dosage). All other anti-allergic medications were then discontinued and the time to flare (TTF) needing anti-allergic treatment was determined.

Inclusion criteria. For a dog to be selected for this study, the following three criteria had to be met:

1. It suffered from AD diagnosed from a compatible history and the fulfilment of five published criteria (Favrot C, Steffan J, et al., Vet Dermatol 2010; 21: 23-30). All skin diseases potentially resembling AD had to be ruled out, when relevant, according to diagnostic and treatment standards. We did not exclude dogs with food-induced AD if they remained on the diets known not to cause a flare of AD; and

2. It had had at least one flare of AD in the preceding six months; and

3. At the time of the first lokivetmab injection, AD was considered to be controlled with any combination of standard of care anti-allergic medications (i.e. topical or oral glucocorticoids, ciclosporin, oclacitinib or oral antihistamines). We defined “controlled AD” as one with no or mild pruritus (subjective or Pruritus Visual Analog Scale ≤two of 10 evaluated by the owner), with no or mild cutaneous erythema and no or only focal skin or ear canal infections.

Exclusion criteria. The exclusion criteria for any dog were:

1. It had superficial pyoderma or yeast infection requiring oral antimicrobial medications at the time of the first lokivetmab injection; or

2. Any of the anti-allergic medications defined above were administered for more than four weeks after the first lokivetmab injection; or

3. Allergen-specific immunotherapy had been initiated less than 12 months before the first lokivetmab injection; or

4. Lokivetmab was injected less often than every four weeks if given at a 1 mg/kg dosage or every eight weeks for a 2 mg/kg dosage dosage.

### Treatment Protocol and Follow-Ups:

We used the commercially available lokivetmab in single 1 mL-vials containing either 10, 20, 30 or 40 mg (Cytopoint, Zoetis). Lokivetmab injections were repeated every four weeks for the 1 mg/kg dosage (EU dosage), or four to eight weeks for 2 mg/kg (US dosage); each of the two dosages was selected by clinician and owner after considering the cost and expected interval between injections. All of the enrolled dogs were followed prospectively until the pre-defined study end-point (see next section). Topical antiseptics, shampoos without glucocorticoids, or otic medications (even with a glucocorticoid) could be used, as needed, during the follow-up period.

### End-Point of the Study:

Each dog was followed until a flare of AD occurred, or one year after the last patient was enrolled (30 Mar. 2018), whichever came first. In this study, we defined a flare of AD as an episode resulting in the need for an escalation of anti-allergic treatment (other than lokivetmab) or for seeking additional (veterinary) medical advice, as pro-posed previously for humans with AD (Langan S M, Thomas K S, Williams H C. What is meant by a “flare” in atopic dermatitis? A systematic review and proposal Arch Dermatol 2006; 142: 1,190-1,196; Thomas K S, Stuart B, O'Leary C J et al. Validation of treatment escalation as a definition of atopic eczema flares. PLoS One 2015; 10: e0124770).

### Outcome Measures:

The primary outcome measure was the TTF of AD, defined as the time (in days) elapsed between the flare and the last day of administration of any of the previously given anti-allergic medications. The Kaplan-Meier method was used to estimate the median and distribution of TTF of AD. The secondary outcome measures were the percentages of dogs not having had a flare of AD at three, six, nine and 12 months after the first lokivetmab injection.

### Results:

### Phase 1:

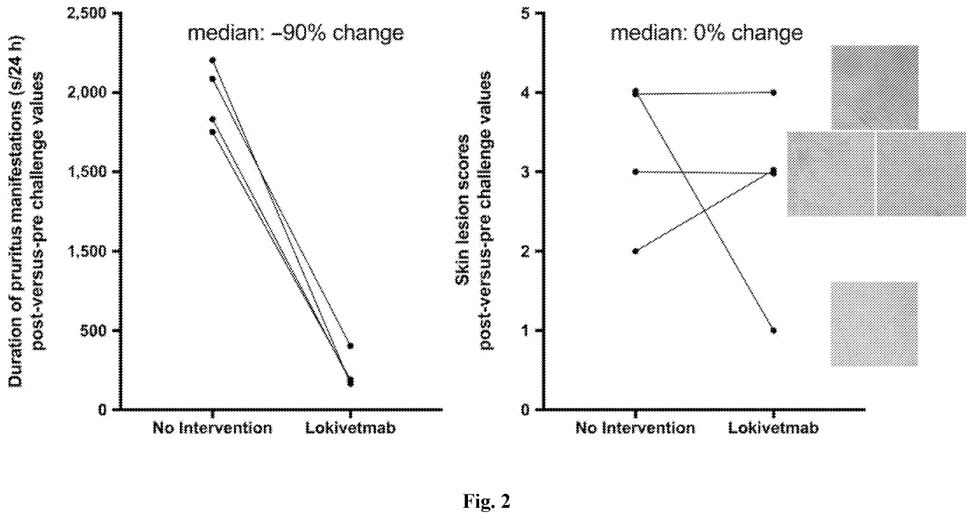

The results of this experiment are presented in Table 1 and FIG. 2. Without any intervention, the application of HDM onto the skin of these sensitized dogs led to a marked increase in pruritus manifestation duration (a median change of over 2,000%) and mild-to-moderate skin lesions with a median SLS of 4 (Table 1).

When these dogs were proactively treated with a single injection of lokivetmab, one week before challenge, and compared with their expected “no intervention” scores, there was a reduction in both pre-challenge DPMs in the 24 h before the allergenic challenge (“pre-challenge”) and in those during the 24 h that followed it (“post-challenge”; Table 1). Compared to results without intervention, the median change in DPM due to the lokivetmab proactive therapy was −90% (range: −81 to −93%; FIG. 2, left panel).

By contrast with the substantial effect on preventing the development of pruritus manifestations, there was little apparent benefit of the lokivetmab proactive therapy to prevent the induction of skin lesions (Table 1, FIG. 2, right panel). The median change in SLS due to lokivetmab proactive therapy was 0% (range: −75% to +50%).

TABLE 1
Comparison of duration of pruritus manifestations and skin lesion scores with or
without lokivetmab proactive therapy.

| | No Intervention | | | | Lokivetmab pretreatment | | | |
|---|---|---|---|---|---|---|---|---|
| | Pre- | Post- | Post- minus − | Post- versus - | Pre- | Post- | Post- minus − | Post- versus - |
| | challenge | challenge | pre | pre (%) | challenge | challenge | pre | pre (%) |
| Duration of pruritic | 107 (52-178) | 2,374 | 2,283 | 2,115 | 35 | 210 | 185 | 601 |
| manifestations | | (2,173-2,580) | (2,090-2,402) | (1,349-4,500) | (20-67) | (205-470) | (165-403) | (413-950) |
| (s/24 h) | | | | | | | | |
| Skin Lesion Scores | 0 (0-0) | 4 (2-4) | 4 (2-4) | Not calculated | 0 (0-0) | 3 (1-4) | 3 (1-4) | Not calculated |

Data presented as median (range).

### Phase 2:

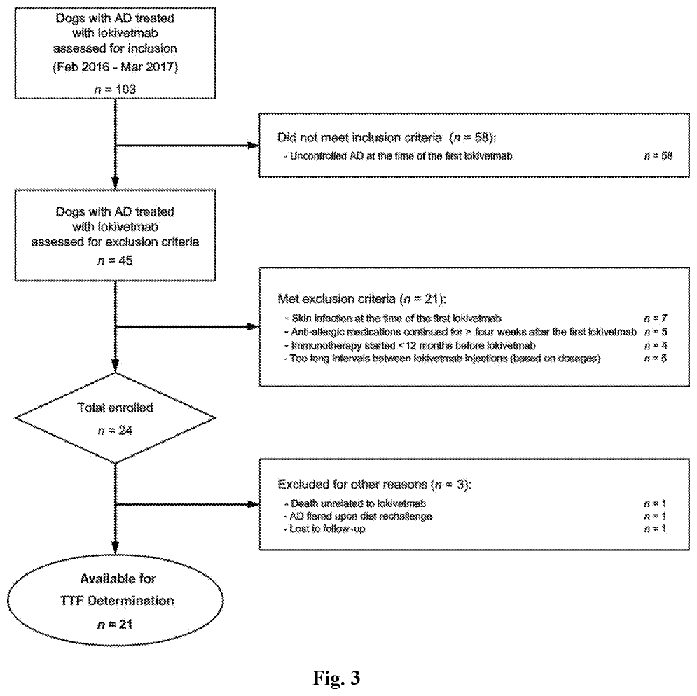

In total, 103 dogs with AD received the first lokivetmab injection at our institution during the recruitment period. Of these 103 dogs, 58 did not meet inclusion criteria, 21 were removed based on one or more exclusion criteria and we excluded three others from analysis due to different reasons (FIG. 3). Therefore, 21 enrolled dogs were followed for up to one year of lokivetmab proactive therapy.

Two dogs (cases 18 and 20) stopped receiving additional lokivetmab injections in spite of not having had a flare of AD during the follow-up period; these dogs were censored (i.e. were not analyzed further as data were incomplete or not informative) from the study on the day of the last lokivetmab injection, but they remained included in the data analysis. Three dogs (cases 5, 6 and 15) did not have a flare of AD by the end of the study; they were included in data analysis with an end-of-study censoring.

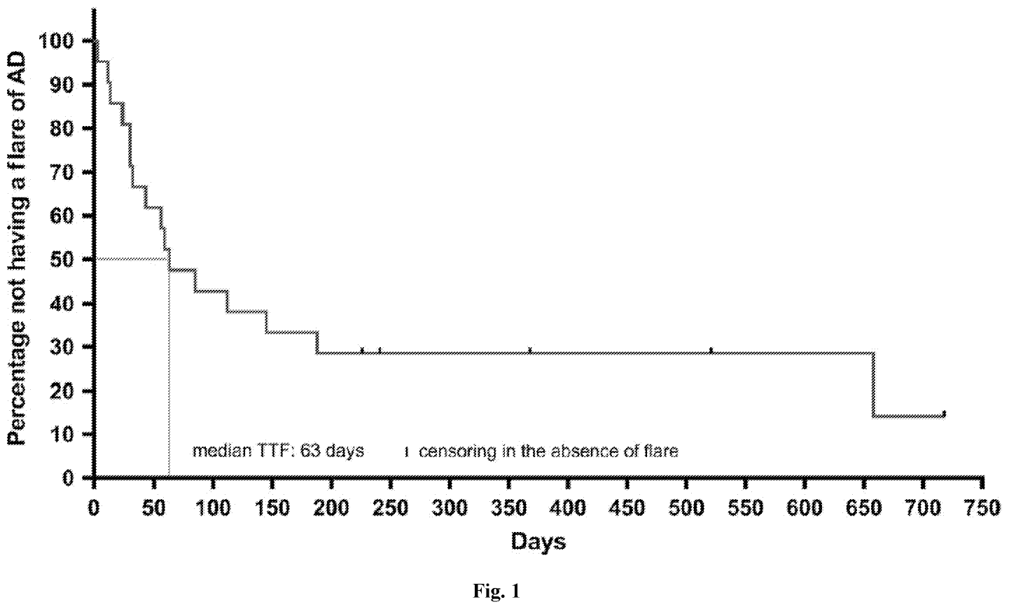

Altogether, the earliest TTF of AD after discontinuation of anti-allergic medications was three days. By contrast, one of the censored dogs (case 5) did not have a flare of AD until the end of the study with a TTF of AD longer than 718 days. The Kaplan-Meier curve, designed to reflect the percentage of dogs not having a flare of AD over time, yielded a median TTF of AD of 63 days (range: 3 to over 718 days) for these 21 dogs (FIG. 1). The observed percentages of dogs not having had a flare of AD at three, six, nine and 12 months was 43% (9 dogs), 33% (7), 19% (4) and 19% (4), respectively. Taking into account the censored dogs in the Kaplan-Meier analysis, the percent-ages of dogs not having had a flare of AD at these time points were, respectively, 43%, 33%, 28% and 28% (FIG. 1). Thus, taking into account dogs censored to loss of follow up, about 30% of the dogs remained free of flare one year after discontinuing all anti-allergic drugs outside the lokivetmab.

In this study, we tested the ability of the anti-IL-31 lokivetmab to prevent the development of clinical signs in an experimental model of acute AD skin lesions in dogs and a prospective follow-up of dogs treated proactively with this mAb.

In our acute canine AD experimental model, we had shown previously that the IL-31-encoding gene was rapidly transcribed after allergen challenge and that this cytokine gene was the most highly upregulated among those coding for the known protein pruritogens examined (Olivry T, Mayhew D, Paps J S et al. Early activation of Th2/Th22 inflammatory and pruritogenic pathways in acute canine atopic dermatitis skin lesions. J Invest Dermatol 2016; 136: 1,961-1,969). In the current study, we confirmed that IL-31 was uniquely responsible for acute atopic itch, because a single lokivetmab injection prevented most of the HDM-induced pruritus manifestations. By contrast, blocking this cytokine alone did not prevent the development of acute erythematous lesions. Because skin prick tests with IL-31 induced a long-lasting erythema in healthy and atopic humans (Hawro T, Saluja R, Weller K et al. Interleukin-31 does not induce immediate itch in atopic dermatitis patients and healthy controls after skin challenge. Allergy 2014; 69: 113-117), we had expected that inhibiting this cytokine would have had a higher effect to prevent the visible allergen-induced acute skin inflammation. Nevertheless, while not wishing to be bound to any particular theory, our results suggest a minimal role for this cytokine, or mediators derived from it, in the generation of early AD skin lesions that follows an epicutaneous allergen challenge. This observation of skin inflammation development not blocked by lokivetmab is noteworthy. Indeed, if it were not treated immediately and were it to occur over a large area of skin, one could assume that it might evolve into a bona fide disease flare involving numerous inflammatory mediators and cells, epidermal hyperplasia and an inflammation-induced stratum corneum barrier defect with resulting skin surface microbial dysbiosis, thus needing a multimodal therapy and/or broadly targeting anti-allergic drugs.

Similarly to our results, the “proactive” treatment of NC/Nga atopic mice with an anti-IL-31 mAb reduced the scratching behavior but did not improve clinical dermatitis scores or microscopic changes in skin biopsies com-pared to those of albumin control-injected mice. In that study of atopic mice (Grimstad O, Sawanobori Y, Vestergaard C et al. Anti-interleukin-31-antibodies ameliorate scratching behavior in NC/Nga mice: a model of atopic dermatitis. Exp Dermatol 2009; 18: 35-43), the mean dermatitis score continued to increase despite repeated treatment with the anti-IL-31 mAb every fifth day for seven weeks, thereby corroborating the relative lack of importance of IL-31 in atopic skin inflammation. Finally, our results mirror those seen when inducing allergic contact dermatitis in IL-31-deficient (Il31−/−) mice (Takamori A, Nambu A, Sato K et al. IL-31 is crucial for induction of pruritus, but not inflammation, in contact hypersensitivity. Sci Rep 2018; 8: 6639). In this model as in ours, whereas the allergic pruritus was reduced after hapten application, the local skin inflammation was not. The results seen with proactive lokivetmab therapy match those obtained after the “reactive” treatment of dogs or humans with active AD with—respectively—lokivetmab or the anti-IL-31 receptor nemolizumab. Both of these mAbs lead to a lower reduction of skin lesion than pruritus scores, thereby confirming the minor role of IL-31 in atopic inflammation compared to allergic itch in both species (Michels G M, Ramsey D S, Walsh K F et al. A blinded, randomized, placebo-controlled, dose determination trial of lokivetmab (ZTS-00103289), a caninized, anti-canine IL-31 monoclonal anti-body in client owned dogs with atopic dermatitis. Vet Dermatol 2016; 27: 478-e129; Moyaert H, Van Brussel L, Borowski S et al. A blinded, randomized clinical trial evaluating the efficacy and safety of lokivetmab compared to ciclosporin in client-owned dogs with atopic dermatitis. Vet Dermatol 2017; 28: 593-e145; Ruzicka T, Hanifin J M, Furue M et al. Anti-interleukin-31 receptor A antibody for atopic dermatitis. N Engl J Med 2017; 376:826-835).

In the second phase of our study, the prospective follow-up of client-owned dogs, half of the dogs treated proactively with monthly to bimonthly lokivetmab injections had a flare of AD requiring treatment escalation within the first two months after discontinuing all anti-allergic drugs. Even though a potential limitation of this study is that it was not controlled with a placebo, a possible unethical intervention in this situation where dogs had a controlled disease when they were selected, we chose to compare our TTFs with those generated in two previously published trials that have evaluated AD flares after standard-of-care anti-allergic drug discontinuation. In the first study (Steffan J, et al. Vet Rec 2004; 154: 681-684), 100 dogs were treated for four months with tapering doses of either oral ciclosporin (Elanco; Greenfield, Ind., USA) or methylprednisolone (Oro-Medrol, Zoetis). The median TTFs after ciclosporin and methylprednisolone discontinuation, estimated from the published Kaplan-Meier figure, were 44 and 26 days, respectively. In the second study (Lourencjo A M, Schmidt V, Sao Braz B et al. Efficacy of proactive long-term maintenance therapy of canine atopic dermatitis with v0.0584% hydrocortisone aceponate spray: a double-blind placebo controlled pilot study. Vet Dermatol 2016; 27: 88-92. e25), 41 dogs were treated with an HCA spray (Cortavance, Virbac) and then, once signs were in remission or comparable to those of mild AD, they were treated with weekend applications of proactive HCA or placebo. The median TTFs after placebo and HCA proactive therapy were 33 and 112 days, respectively. Although these result cannot be compared directly because of differences in canine populations and study designs, the TTF that we observed after lokivetmab proactive therapy seems to be about twice that seen without any treatment, but only half that obtained with twice-weekly proactive topical glucocorticoid therapy (Steffan J, Horn J, Gruet P et al. Remission of the clinical signs of atopic dermatitis in dogs after cessation of treatment with cyclosporin A or methylprednisolone. Vet Rec 2004; 154: 681-684). Furthermore, we compared our post-lokivetmab Kaplan-Meier TTF curve with those of the HCA proactive study using a partial dataset provided by the authors of that study; the TTFs after lokivetmab therapy were significantly longer than those after placebo but not after the HCA (data not shown). We suspect that, compared to the topical HCA, the shorter median TTF seen after lokivetmab is likely due to both a broader anti-allergic effect of the glucocorticoid and a limited role of IL-31 in the inflammation of atopic skin.

In humans with AD, there is no clear consensus for how long patients need to be treated with anti-allergic drugs (i.e. during induction therapy until the control of not only macroscopic lesions, but also subclinical inflammation) before moving onto a phase of proactive maintenance therapy (Tang T S, Bieber T, Williams H C. Are the concepts of induction of remission and treatment of subclinical inflammation in atopic dermatitis clinically useful? J Allergy Clin Immunol 2014; 133: 1,615-1,625). An “induction of remission” phase that is too short likely leads to a failure of managing AD. In our study, we defined dogs with “controlled AD” as those having no or mild pruritus, no or mild erythema and no or focal skin/ear infection; such dogs were then selected to be treated proactively with lokivetmab maintenance therapy. While not wishing to be bound to any particular theory, the rapid flares seen in some dogs after discontinuation of the anti-allergic drugs, less than two weeks in three of our dogs, suggest that inflammation was still present at the time of standard treatment cessation and that a longer induction of anti-allergic drugs might have been needed in these patients.

Another possibility for the relatively poor flare-preventive efficacy of proactive therapy with lokivetmab is a shift in the cytokine repertoire during the evolution of AD skin lesions. In canine as in human AD, although Th2-type cytokines are involved in acute lesions, a more complex cytokine milieu involving—at least—a Th1 response is believed to predominate during chronic stages (Pucheu-Haston C M, Bizikova P, Marsella R et al. Review: lym-phocytes, cytokines, chemokines and the T-helper 1-T-helper 2 balance in canine atopic dermatitis. Vet Dermatol 2015; 26: 124-e32; Gavrilova T. Immune dysregulation in the pathogenesis of atopic dermatitis. Dermatitis 2018; 29: 57-62). Interleukin-31 being one of the Th2 cytokines, it would be logical to expect that the lesion-preventive effect of lokivetmab might decrease after the acute lesions begin to mature and the cytokine repertoire shifts away from a Th2-predominant one. Indeed, during a flare of canine AD lesions induced by an allergen challenge in HDM-sensitized atopic dogs, there is a variable cytokine expression over time, even in the first two days after provocation, thereby likely diluting the importance of IL-31 as lesions progress (Marsella R, Olivry T, Maeda S. Cellular and cytokine kinetics after epicutaneous allergen challenge (atopy patch testing) with house dust mites in high-IgE beagles. Vet Dermatol 2006; 17:111-120; Olivry T, Mayhew D, Paps J S et al. Early activation of Th2/Th22 inflammatory and pruritogenic pathways in acute canine atopic dermatitis skin lesions. J Invest Dermatol 2016; 136: 1,961-1,969). Finally, in the current model of the cytokine cascade characteristic of AD inflammation, IL-31 is secreted downstream from several other cytokine groups, including, for example, the keratinocyte-derived cytokines IL-33, thymic stromal lymphopoietin (TSLP), IL-25 and IL-17C and the inflammatory cell-secreted Th2 cytokines IL-4, IL-5 and IL-13 (Gavrilova T., Dermatitis 2018; 29: 57-62; Weidinger S, et al., Nat Rev Dis Primers 2018; 4: 1-018-0001-z; and Guttman-Yassky E, Krueger JG. J Invest Dermatol 2018; 138: 1,467-1,469). As a result, even if lokivetmab were to block the action of IL-31 completely, there would be ample opportunity for inflammation to develop due to the action of the many mediators secreted upstream of, or concurrently with, IL-31.

In spite of the flare of AD seen in half of the case study dogs in the first two months of proactive lokivetmab monotherapy, over a quarter of followed cases did not exhibit a disease flare for more than one year. Although this percentage does not seem high and while not wishing to be bound to any particular theory, because AD is a multifactorial disease that requires a multi-faceted treatment approach (Olivry T, DeBoer D J, Favrot C et al. Treatment of canine atopic dermatitis: 2015 updated guidelines from the international committee on allergic diseases of animals (ICADA). BMC Vet Res 2015; 11: 210), the long-term remission seen in some dogs after targeting a single cytokine suggests that IL-31 is critically involved in the pathogenesis of clinical signs of AD in these cases.

Finally, we would be remiss in not mentioning that, in humans, different phenotypes of AD have been reported to occur in different ethnic groups. For example, Asian patients with AD have an increased epidermal hyperplasia and parakeratosis and a higher Th17 activation compared to the European American atopics (Noda S, Suarez-Farinas M, Ungar B et al., J Allergy Clin Immunol 2015; 136: 1,254-1,264). Similarly, the early onset paediatric human AD also exhibits a robust Th17 response (Brunner PM, et al. Early-onset pediatric atopic dermatitis is characterized by TH2/TH17/TH22-centered inflammation and lipid alterations J Allergy Clin Immunol 2018; 141: 2,094-2,106). The reported variation of clinical phenotypes among atopic dogs of different breeds (Wilhem S, Kovalik M, Favrot C. Breed-associated phenotypes in canine atopic dermatitis. Vet Dermatol 2011; 22: 143-149) could be interpreted as different genetically influenced pathomechanisms being present in canine as in human AD. Such variation in breed-associated pathogenesis—and perhaps even one related to the age at the time of treatment—could potentially lead to a heterogeneity in the efficacy of lokivetmab proactive therapy. As we only had a small number of dogs of different breeds, a breed- or age-specific response to lokivetmab prevention therapy could not be evaluated.

We showed herein that proactive maintenance therapy of canine AD with lokivetmab injections led to a long-lasting remission of clinical signs in some cases. While not wishing to be bound to any particular theory, these results suggest the existence of a heterogeneity in lesion development mechanism in dogs as in humans with AD (that is the existence of “different endotypes”), and that factors other than IL-31 are involved in AD skin lesion development in most dogs with AD.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

1. A method of preventing, reducing, and/or inhibiting an allergic disorder in a subject in need thereof, the method comprising administering to the subject a composition comprising a therapeutically effective amount of a biologic agent targeting an inflammatory mediator before and/or during an early phase of inflammation, with the proviso that the biologic agent is not an anti-IgE antibody.

2. The method of claim 1, wherein the subject is asymptomatic or paucisymptomatic with or without currently receiving and/or having received an anti-inflammatory agent, an anti-allergy agent, an immunomodulatory agent or a combination thereof

3. The method of claim 2, wherein the anti-inflammatory agent, the anti-allergy agent, and/or the immunomodulatory agent is selected from a glucocorticoid, a non-steroidal anti-inflammatory agent, a leukotriene antagonist, a JAK inhibitor, an immunoglobulin, an anti-histamine, allergen-specific immunotherapy agent, non-specific immunotherapy agent, and combinations thereof administered by either oral, subcutaneous, intramuscular, intravenous and/or topical routes.

4. The method of claim 1, wherein the subject is a canine, feline, equine, or human.

5. The method of claim 4, wherein the subject is a human.

6. The method of claim 1, wherein the inflammatory mediator is selected from histamine, a leukotriene, a prostaglandin, a cytokine, platelet activating factor (PAF), a growth factor, a protease, an immunoglobulin, and combinations thereof.

7. The method of claim 6, wherein the inflammatory mediator is a cytokine selected from interleukin-4, interleukin-5, interleukin-6, interleukin-9, interleukin-10, interleukin-13, interleukin-22, interleukin-25, interleukin-31, interleukin-33, interleukin-4 receptor, interleukin-5 receptor, interleukin-6 receptor, interleukin-9 receptor, interleukin-10 receptor, interleukin-13 receptor, interleukin-22 receptor, interleukin-25 receptor, interleukin-31 receptor, interleukin-33 receptor, thymic stromal lymphopoietin (TSLP), thymic stromal lymphopoietin receptor, tumor necrosis factor alpha, tumor necrosis factor alpha receptor, and combinations thereof.

8. The method of claim 1, wherein the allergic disorder comprises allergic inflammation and/or chronic inflammation.

9. The method of claim 8, wherein the allergic disorder comprises allergic inflammation and is selected from allergic rhinitis, atopic dermatitis, allergic asthma, allergic conjunctivitis, gastro-intestinal inflammation, urticarial, latex allergy, and/or food allergy.

10. The method of claim 9, wherein the allergic disorder is atopic dermatitis.

11. The method of claim 10, wherein the subject has received and/or is currently receiving, cyclosporine, glucocorticoids, oclacitinib, by any route, or a combination thereof.

12. The method of claim 8, wherein the allergic disorder is celiac disease, vasculitis, chronic obstructive pulmonary disease (COPD), irritable bowel disease (IBS), atherosclerosis, arthritis, systemic lupus erythematosus, multiple sclerosis, asthma, chronic peptic ulcer, sinusitis, tuberculosis, rheumatoid arthritis, periodontitis, ulcerative colitis, Crohn's disease, atopic dermatitis (eczema), rosacea, seborrheic dermatitis, and/or psoriasis.

13. The method of claim 1, wherein the biologic agent is a monoclonal antibody and/or a decoy receptor and/or a vaccine that induces the production of an antibody or a decoy receptor.

14. The method of claim 13, wherein the monoclonal antibody is an anti-interleukin-31 receptor A antibody, an anti-interleukin-4 receptor alpha antibody, an anti-IL-13 antibody, an anti-IL-22 antibody, an anti-TSLP antibody, an anti-IL-31 antibody, and/or an anti-IL-33 antibody, optionally wherein the monoclonal antibody is an anti-IL-33 antibody.

15. The method of claim 14, wherein the monoclonal antibody is lokivetmab, nemolizumab, or dupilumab.

16. The method of claim 1, wherein the biologic is administered about every 1 week to about every 8 weeks.

17. The method claim 1, wherein the biologic is administered in combination with an anti-infectious agent.

18. The method of claim 17, wherein the anti-infectious agent is an anti-septic, an antibiotic, an antifungal, or a combination thereof.

19. The method of claim 1, wherein the allergic disorder is atopic dermatitis (eczema), rosacea, seborrheic dermatitis, and/or psoriasis and the biologic is administered in combination with topical moisturizers and/or baths.

20. (canceled)

21. The method of claim 19, wherein the method provides reduced or delayed flares, relapses and/or recurrences of lesions in a subject with atopic dermatitis.
